(19) United States
(12) Patent Application Publication
(10) Pub. No.: US 2025/0344659 A1
(43) Pub. Date: Nov. 13, 2025

(54) SEXUAL KENTUCKY BLUEGRASS CULTIVARS AND BREEDING STRATEGIES

(71) Applicant: NEXGEN Plant Science Center, LLC, Jefferson, OR (US)

(72) Inventors: Kenneth Hignight, Albany, OR (US); Debra Hignight, Albany, OR (US); Joseph Wipff, Albany, OR (US)

(73) Assignee: NEXGEN Plant Science Center, LLC, Jefferson, OR (US)

(21) Appl. No.: 19/216,147

(22) Filed: May 22, 2025

Related U.S. Application Data
(62) Division of application No. 18/233,718, filed on Aug. 14, 2023.
(60) Provisional application No. 63/397,699, filed on Aug. 12, 2022.

(51) Int. Cl. A01H 6/46; A01H 5/10

(52) U.S. Cl. CPC A01H 6/466; A01H 5/10

(57) ABSTRACT

A method for producing 100% sexual cultivars of a grass plant developed from facultative apomictic germplasm. Sexual cultivars of Kentucky bluegrass include those of breeder's codes: AKB020, AKB413, AKB419, AKB484, AKB357, and AKB397. Additionally, disclosed is a method for producing hybrid apomictic plants from hybridization of a completely sexual parent with a facultative or apomictic parent, including those of breeder's codes AKB1661, AKB1658, AKB3605, AKB2839, AKB2949, AKB3128, AKB3179, AKB3201, AKB3216, AKB3241.

### CROSS-REFERENCE TO RELATED APPLICATION(S)

This application is a divisional of U.S. patent application Ser. No. 18/233,718, filed Aug. 14, 2023, and entitled “Sexual Kentucky Bluegrass Cultivars and Breeding Strategies”, which claims the benefit under 35 U.S.C. § 119 (e) to U.S. Provisional Application 63/397,699, filed Aug. 12, 2022, and entitled “Development of Sexual Kentucky Bluegrass Cultivars,” each of which are hereby incorporated herein by reference in its entirety for all purposes.

### TECHNICAL FIELD

The disclosure relates to grass cultivars and breeding strategies therefor. In particular to Kentucky Bluegrass cultivars capable of 100% sexual reproduction and methods of producing apomictic cultivars.

### BACKGROUND

Reproduction in plants is ordinarily classified as sexual or asexual. Sexual reproduction is accomplished in plants basically the same way it occurs in other sexually reproducing organisms. Sexual reproduction produces offspring by the fusion of gametes, a sperm (pollen formed in the anther) fertilizes the egg (in the ovary), resulting in offspring genetically different from the parent or parents. This sexual reproduction creates seed which is the next generation even before sprouting. In sexual reproduction, usually a megaspore mother cell arising from the hypodermal layer of the ovule enlarges and goes through meiosis, and two cell divisions, to form a linear tetrad of megaspores each with a haploid chromosome number. The three micropylar spores degenerate while a functional chalazal spore enlarges to form an embryo sac with an egg, two polar nuclei, two synergids, and three antipodals.

The Normal or Polygonum type of embryo sac formation is characteristic of practically all grass (Poaceae) genera, including Poa L. The embryo sac is a seven-celled, eight-nucleate structure; each cell has a different form of structural specialization. The whole embryo sac is polarized from the basal (or chalazal) end to the proximal (or micropylar) end. At the micropylar (proximal) end are three cells—a highly vacuolated egg cell and two synergids—that together are called the egg apparatus. In the middle of the ovule is the central cell, a large, vacuolated cell containing many organelles and two nuclei (polar nuclei) that fuse fully or partially prior to fertilization. At the chalazal end of the ovule are three antipodal cells. Fertilization involves a pollen tube entering the ovule through the micropylar opening and releasing its sperm cells. An attractant released by the synergid cells guides the pollen tube to the female gametophyte. Fusion of one sperm cell with the egg cell forms a diploid zygote, and fusion of the other sperm cell with the central cell forms a triploid endosperm (double fertilization).

The term apomixis is generally accepted as the replacement of sexual reproduction by various forms of asexual reproduction. Apomixis is a genetically controlled method of reproduction in plants where the embryo is formed without union of an egg and a sperm. There are three basic types of apomictic reproduction: 1) apospory (somatic apomixis), the megagametophyte (female gametophyte) arises from some other somatic nucleus cell. The megagametophyte develops from a chromosomally unreduced egg in an embryo sac derived from a somatic cell; 2) diplospory (gametophytic apomixis) in which the megagametophyte arises from a cell of the archesporium (the cell or group of cells from which spore mother cells develop; the megaspore mother cell); and 3) adventitious embryony (sporophytic apomixis), an embryo (not from a gametophyte) is formed directly from nucellus or integument tissue. In most forms of apomixis, pseudogamy or fertilization of the polar nuclei to produce endosperm is necessary for seed viability. The main difference in diplospory compared to sexual development is that a single megaspore is produced by the megaspore mother cell, and this megaspore has the somatic chromosome number which results in an embryo sac similar in appearance to a sexual embryo sac but with an egg containing the somatic chromosome number. In adventitious embryony, embryos develop directly from somatic cells of the ovule without formation of embryo sacs. Sexual sacs which are needed for endosperm formation may also form in the same ovule.

Apomixis is a reproductive process that bypasses female meiosis and syngamy (fusion of the two gamete cells, or of their nuclei, in reproduction) to produce embryos genetically identical to the maternal parent. With apomictic reproduction, progeny of especially adaptive or hybrid genotypes maintain their genetic fidelity throughout repeated life cycles.

Apospory is the type of apomixis found in Kentucky bluegrass. In apospory, a megaspore mother cell may begin enlarging and even produce chromosomally reduced megaspores, but this sexual tissue dies and usually degenerates before embryo sac development. Instead, somatic cells of the nucellus enlarge and the nuclei of these cells go through mitotic divisions to form one to many embryo sacs per ovule each with one to eight chromosomally unreduced nuclei. Aposporous apomicts are characterized by the participation of one or more nucellar cells in the direct formation of one or more embryo sacs. Each nucleus of the aposporous embryo sac has the somatic chromosome number and genotype of the maternal plant. Most aposporous apomictic species are pseudogamous, in that they require pollination and fertilization of polar nuclei for the development of endosperm, but the unreduced aposporous egg develops without fertilization (parthenogenetically). Female meiosis usually is disturbed in aposporous apomicts that form all of their seed asexually (obligate apomicts) so that no functional megaspore continues development beyond the first mitotic division.

Kentucky bluegrass (Poa pratensis L.) is a widely propagated turfgrass species used in the Northern Hemisphere. It is also extensively cultivated worldwide as a forage grass. Kentucky bluegrass is a widely adapted and highly variable long-lived perennial species which spreads not only by seeds, but also by its extensive determinate rhizomes. Most, if not all, of the naturalized germplasm growing in the United States is the result of introduction from Europe. Kentucky bluegrass is classified as a facultative apomictic species. In facultative apomicts, the individual genotypes reproduce both asexually and sexually, controlled by genetic mechanisms which are influenced by environmental parameter so that there is a range in expression of both of the methods of reproduction.

It has been noted that nearly every characteristic needed for an ideal lawn is present in Kentucky bluegrass. These characteristics include enhanced tolerance to drought, heat, shade, close mowing, excessive wear, acid soils, salinity, as well as many turfgrass diseases. The art currently lacks the necessary techniques required to combine the characteristics from various genotypes into a single cultivar.

There is a need in the art to provide breeding techniques to access the genetic variation in Kentucky bluegrass made inaccessible by apomixis

### BRIEF SUMMARY

Disclosed herein is a method of breeding for completely sexually reproducing cultivars from an apomictic, asexually reproducing, species, Poa pratensis L., as well as the plant parts, including the seed. Methods of using the sexual lines in further breeding (e.g., selection for disease and other biotic and abiotic stresses) are also provided, as well as breeding strategies to then produce highly apomictic cultivars from sexual populations. Sexual Poa pratensis cultivars and their apomictic derivative cultivars are suitable for use in lawns, golf courses, sod and other turfs. Further disclosed is a method for producing hybrid seed by transferring the apomictic mechanism from the apomictic parent to the progeny from a sexual x apomictic hybridization. Still further disclosed are apomictic turf type cultivars of Kentucky bluegrass developed from the hybridization of 100% sexual cultivars with obligate/facultative apomictic germplasm of Kentucky bluegrass.

In Example 1, a seed of a sexual Kentucky bluegrass capable of 100% sexual reproduction.

Example 2 relates to a grass plant produced by growing the seed of Example 1.

Example 3 relates to a grass plant having all the physiological and morphological characteristics of the grass plant of Example 2.

In Example 4, a method of producing seed capable of 100% sexual reproduction from seed of plants with facultative sexual or apomictic reproduction, comprising: growing plants with facultative sexual or apomictic reproduction; selecting plants with phenotypes of interest; harvesting the selected plants individually; growing the selected plants individually alongside clones of a maternal parent of the selected plants; and determining the percentage of sexual reproduction.

Example 5 relates to the method of Example 4, further comprising crossing one of the selected plants with itself for confirming the percentage of sexual reproduction.

Example 6 relates to the method of any of Examples 4-5, wherein if the percentage of sexual reproduction is less than 100% the method further comprises selecting plants with phenotypes of interest from the previously selected plants, harvesting those plants individually, growing out those harvested plants alongside clones of their maternal parent plant.

Example 7 relates to the method of any of Examples 4-6, wherein the plants are Kentucky Bluegrass.

Example 8 relates to a seed of a plant produced via the method of any of Examples 4-7.

Example 9 relates to a grass plant produced by growing the seed of Example 8.

Example 10 relates to a grass plant having all the physiological and morphological characteristics of the grass plant of Example 9.

In Example 11, a method for producing hybrid seed comprising: crossing a maternal completely sexual plant with a paternal facultative or apomictic cultivar to create a first hybrid generation; growing the first hybrid generation; selecting phenotypes of interest from plant of the first hybrid generation; harvest selected phenotype plants individually; and grow individually the selected phenotype plants along side a clone of a maternal parent of the select phenotype plant to create a second hybrid generation.

Example 12 relates to the method of Example 11, further comprising determining the percent sexuality of the second hybrid generation.

Example 13 relates to the method of any of Examples 11-12, further comprising releasing a cultivar for the second hybrid generation when the second hybrid generation is apomictic.

Example 14 relates to the method of any of Examples 11-13, wherein the maternal completely sexual plant is an obligate sexual Kentucky bluegrass.

Example 15 relates to the method of any of Examples 11-14, wherein the paternal facultative cultivar is a plant having desired characteristics.

Example 16 relates to the method of any of Examples 11-15, wherein the apomictic mechanism from the paternal facultative or apomictic cultivar is transferred to the first and second hybrid generations.

Examples 17 relates to the method of any of Examples 11-16, wherein the paternal facultative or apomictic cultivar is an apomictic cultivar.

Example 18 relates to a seed of a plant produced via the method of any of Examples 11-17.

Example 19 relates to a grass plant produced by growing the seed of Example 18.

Example 20 relates to a grass plant having all the physiological and morphological characteristics of the grass plant of Example 19.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a schematic diagram showing the frequency of the three methods of reproduction found commonly found in apomictic species (e.g., Kentucky bluegrass), according to one implementation.

FIG. 2 shows a flow diagram for the production of completely sexual species of grass from facultative sexual or apomictic plants, according to one implementation.

FIG. 3 sows a flow diagram for developing apomictic cultivars from completely sexual plants, according to one implementation.

FIG. 4 shows a square cross design table for crossing various cultivars, according to one implementation.

### DETAILED DESCRIPTION

This present invention provides a method for producing 100% sexual cultivars of Kentucky bluegrass developed from facultative apomictic germplasm of Kentucky bluegrass. The 100% sexual cultivars of Kentucky bluegrass (including, but not exclusive to) known as breeder's codes AKB020, AKB413, AKB419, AKB484, AKB357, and AKB397 and methods used to produce the grass are provided.

A further object of the present invention is to provide a method for producing hybrid seed by transferring the apomictic mechanism from the apomictic parent to the progeny from a sexual x apomictic hybridization. Apomictic turf type cultivars of Kentucky bluegrass developed from the hybridization of 100% sexual cultivars with obligate/facultative apomictic germplasm of Kentucky bluegrass (including, but not exclusive to) known as breeder's codes: AKB1661, AKB1658, AKB3605, AKB2839, AKB2949, AKB3128, AKB3179, AKB3201, AKB3216, AKB3241, AKB2403, AKB2313, and AKB2404, and methods used to produce the grass are provided.

It is generally appreciated that hybridization to increase genetic variability for subsequent selection is an important component in plant breeding. Provided herein is a technique to develop completely sexually reproducing populations in order to access the diversity of beneficial characteristics that has been inaccessible in the past in asexually reproducing species. Also described are techniques to develop cultivars from the sexually developed populations, such that vast genetic diversity can be accessed. Additionally described herein are techniques of using these sexual populations in breeding with apomictic plants to develop apomictic cultivars with characteristics derived from the sexual populations.

The various methods described herein provide methods of developing 100% sexual genotypes. These sexual genotypes can be used for the selection of improved traits and then to develop unique improved cultivars, as will be described further herein. 100% sexual genotypes can be used in hybridization programs to develop unique completely sexual cultivars. Further 100% sexual genotypes can be used as a female parent and hybridize it with highly apomictic cultivars as a way to provide the way of returning back to a highly apomictic cultivar for cultivar development.

In Kentucky bluegrass three methods of reproduction are possible: 1) Obligate sexual (very rare in natural world), no apomictic reproduction is present; 2) Facultative apomixis (most common method), in which meiosis and aposporous development occur simultaneously in same plant and both reduced and unreduced embryo sacs ultimately reside in the same individual (meaning that in an inflorescence, individual flowers will either be sexual or apomictic), this is the most common method of reproduction; in rare instances the same ovule will have both reduced and unreduced embryo sacs (an individual ovule with have both a sexual and a single apomictic embryo sac); and 3) obligate apomictic, no sexual embryo sacs ever present (rare).

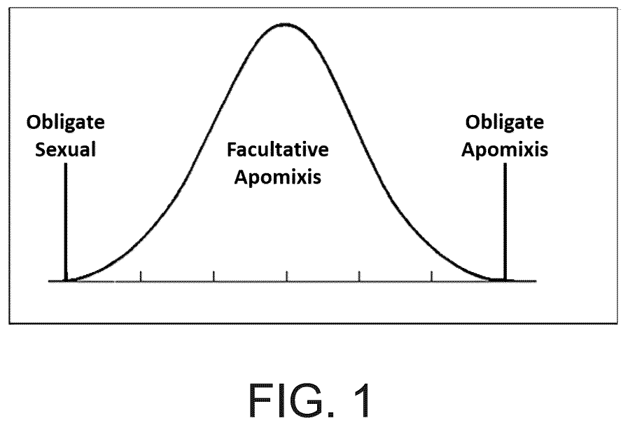

The most common method of reproduction in apomictic species is facultative apomixis, as can be seen in the curve of FIG. 1. In the facultative method the two modes of reproduction, sexual and asexual, coexist in the same individual. Within an individual ovule the following is possible: 1) a sexual embryo sac; 2) a sexual embryo sac and an apomictic embryo sac; 3) a sexual embryo sac and multiple apomictic embryo sac; 4) a single apomictic embryo sac (and no sexual embryo sac); and 5) multiple apomictic embryo sacs (no sexual embryo sac). The frequency range of sexual and apomictic embryo sacs is genetically controlled, but environmentally influenced within an individual genotype to give a range of the percent sexual/apomictic embryo sacs possible in an individual genotype.

If an individual is completely (obligate) apomictic (i.e. nos. 4 & 5 above) there can be two subcategories: 1) functionally obligate; or 2) genetically obligate. In a functionally obligate apomict, the sexual embryo sac begins to develop, but never reaches maturation or viability, but aborts at some point in its development. As such, while a functionally obligate individual has the genetics to start the development of a sexual embryo sac, it is aborted at some point, so this individual is functionally an obligate apomict, because the sexual reproduction potential is there but is not fulfilled. In a genetically apomict phenotype, there is no indication of any development of a sexual embryo sac. This apomict individual is genetically and thus functionally an obligate apomict.

In Kentucky bluegrass, it is rare to have both a sexual and apomictic embryo sac in the same ovule, it is either one or the other. When the Kentucky bluegrass is apomictic, it is a single apomictic embryo sac that mimics the sexual sac completely. The two types of embryo sacs cannot be differentiated under a microscope. They can only be separated by growing out the seed to determine whether progeny look like the maternal parent (apomictic sac) or segregate from the maternal parent (sexual sac). Counts can then be made of the phenotype to determine the percent apomixis/sexual reproduction in a specific genotype. This means there is at least one year in between each cross or selection to determine the percent apomixis/sexuality.

As described herein it is valuable to be able to 1) readily hybridize cross-compatible completely sexual x apomictic genotypes to produce true-breeding F1 hybrids and 2) to develop cultivars directly from sexual genotypes. Sexual genotypes can also be selected for improved agronomic traits and then used to cross with superior apomictic parents and lock in the hybrid vigor in the progeny with apomixis or further develop into a cultivar. The required obligate sexual genotype is extremely rare in nature, and the ones that have been discovered are very weak, unstable and do not persist/live long. Further, the sexual genotypes found in nature are not true breeding (very heterozygous) and except for the extremely rare obligate ones, are facultative apomicts with a high percentage of sexuality versus apomixis.

It would be understood, in light of this disclosure, that apomixes, seeds with apomictic traits, have economic potential because they can cause any genotype, regardless of how heterozygous, to breed true, and thus capture and maintain hybrid vigor. In addition to fixing hybrid vigor, apomixis can make possible commercial hybrid production in crops where efficient male sterility or fertility restoration systems for producing hybrids are not known or developed. As would be appreciated, apomixis can make hybrid development more efficient. It also simplifies hybrid production and increases genetic diversity in plant species with good male sterility systems.

With apomixis removed, then an enormous amount of untapped variation is now available for selection and crop improvement. Though high levels apomixis (>96%) is an excellent means of maintaining genetic purity of a cultivar from one generation to the next, it also makes the hybridization of different genotypes and subsequent selection of improved characteristics a difficult, extremely slow, and often frustrating the breeding process.

Described herein is the ability to develop Kentucky bluegrass populations that are completely sexual, with improved agronomic traits and performance, from improved facultative apomictic populations. The various cultivars and related methods described herein overcome the various problems of the prior art by producing sexual populations of commercial cultivars and improved genetic lines of sexual reproduction populations of Kentucky bluegrass.

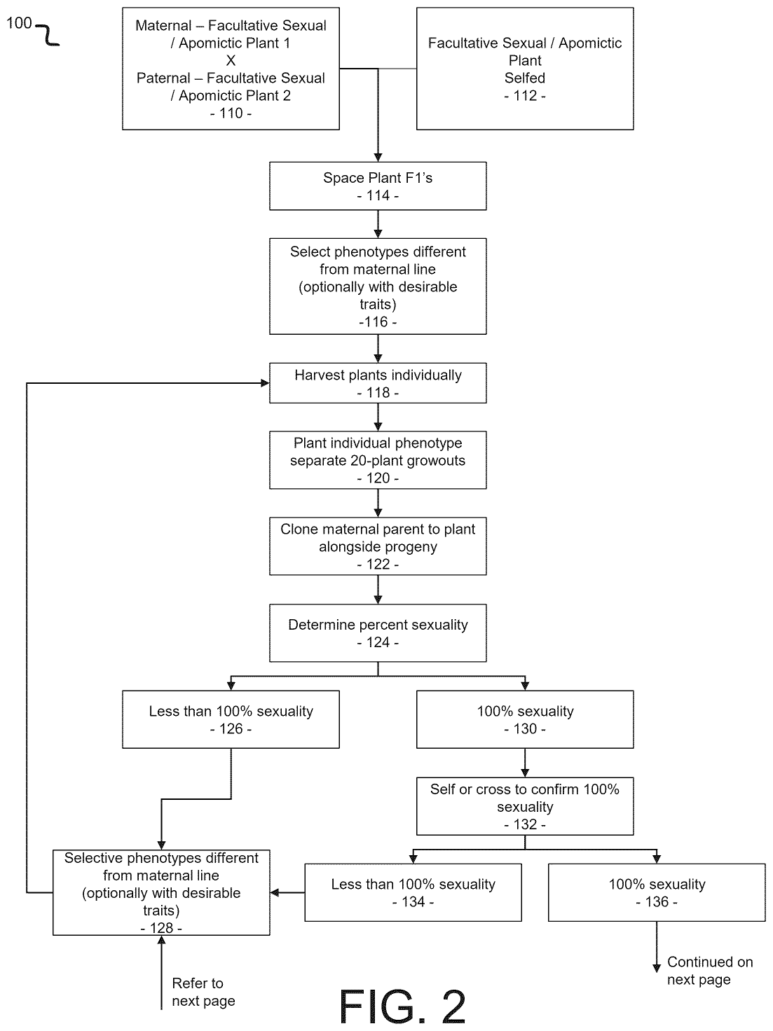

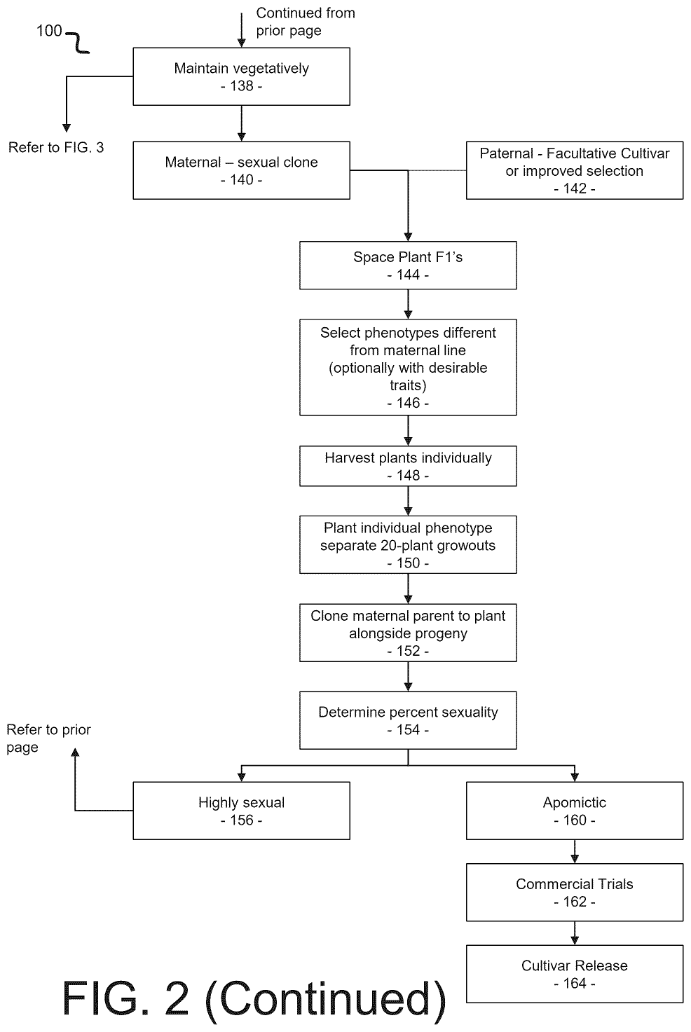

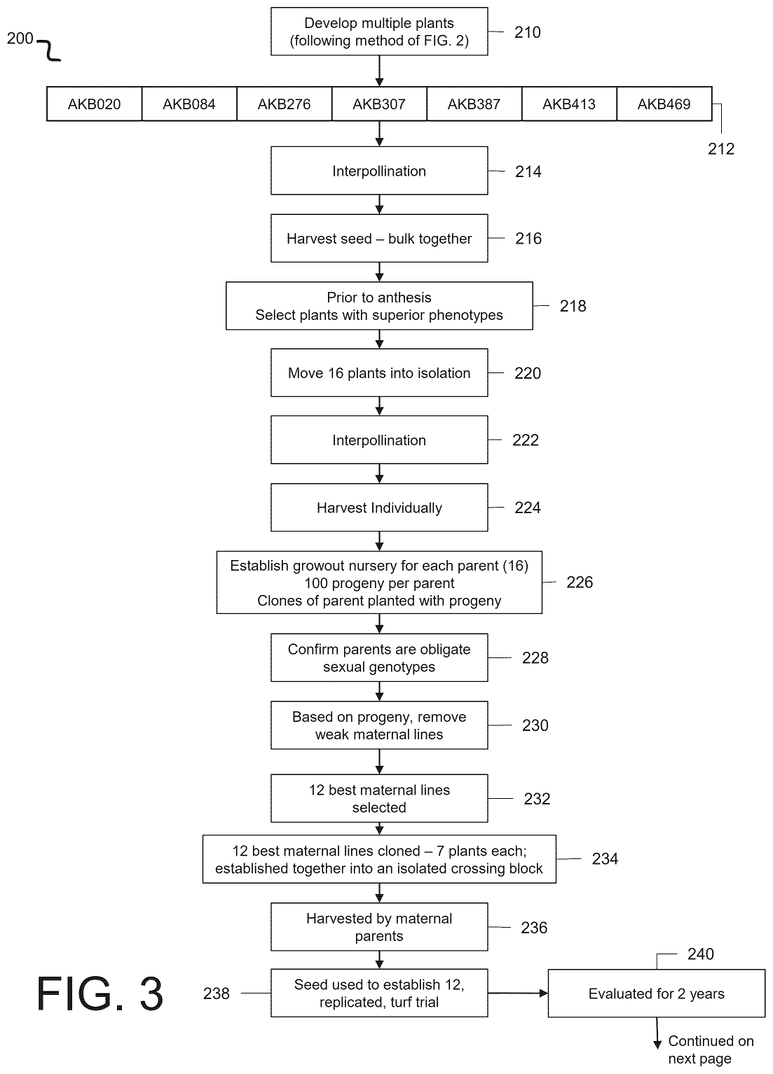

FIG. 2 shows a process diagram of breeding to create completely (100%) sexual lines and subsequent crossing to create hybrids that are apomictic. The process and method described herein is described as a series of steps and substeps, each of which is optional and may be performed in various orders, as would be understood. Certain steps may be performed in sequence, concurrently, iteratively, or not at all. Various additional and/or alternative steps are possible and would be appreciated by those of skill in the art.

In a first optional step, a (maternal) facultative sexual or apomictic plant is crossed with a (paternal) facultative sexual or apomictic plant (box 110). This plant from box 110 is then crossed with a clone of a facultative sexual or apomictic plant (box 112) to create an F1 plant. The F1 plant is then established in a space plant nursery (box 114).

In a further optional step, the phenotypes in the F1 plants different from the maternal line are selected (box 116). Optionally those plants/phenotypes with desirable traits, or traits of interest are selected (box 116). The selected phenotypes/plants are then harvested individually (box 118).

In another optional step, the individual plants selected are planted separately (box 120). For example, each selected phenotype is planted in a 20 plant growout (box 120).

In a still further optional step, a clone of the maternal plant is planted alongside the progeny, phenotypes in the previous steps (box 122). The plants in the grow out nursery can then be evaluated to determine the sexuality of the plant and the percentage of sexual plants and apomictic plants determined (box 124).

If the plants are less than 100% sexual (box 126) then those plants with traits different than the maternal line are selected (box 128). Optionally, plants with traits that are desirable or of interest and different than the maternal line are selected (box 128). Once selected the steps of boxes 118-124 can be repeated, optionally iteratively, until the plants are determined (box 124) to be 100% sexual (box 130).

If/when the plants are 100% sexual (box 130), the plants can be selfed or crossed to confirm the plants are 100% sexual (box 132).

In an optional step, if the plants are not confirmed to be 100% sexual the steps of boxes 128 and 118-124 can be repeated, optionally iteratively until the plants are determined (box 124) and confirmed (box 132) to be 100% sexual (box 136).

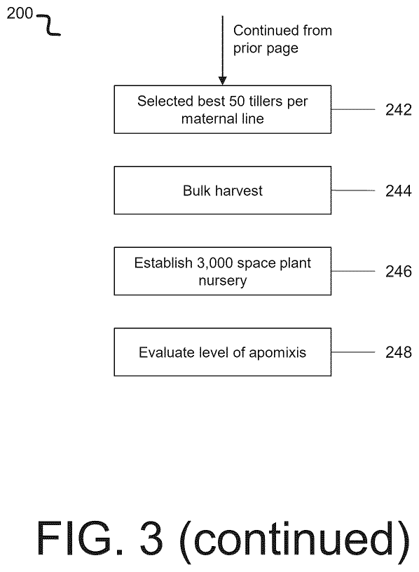

If/when the plants are determined (box 124) and confirmed (box 132) to be 100% sexual (box 136), the plants can be vegetatively maintained (box 138). Optionally, the 100% sexual plants may be used in hybridization, as discussed in reference to FIG. 3 (see boxes 210 and 212).

Continuing with FIG. 2, in a further optional step, a (maternal) sexual clone (box 140) can be crossed with a (paternal) facultative cultivar or improved selection plant (box 142) to create an F1 hybrid. The F1 plant is then established in a space plant nursery (box 144).

In a further optional step, the phenotypes in the F1 plants different from the maternal line are selected (box 146). Optionally those plants/phenotypes with desirable traits, or traits of interest are selected (box 146). The selected phenotypes/plants are then harvested individually (box 148).

In another optional step, the individual plants selected are planted separately (box 150). For example, each selected phenotype is planted in a 20 plant growout (box 150).

In a still further optional step, a clone of the maternal plant is planted alongside the progeny, phenotypes in the previous steps (box 152). The plants in the grow out nursery can then be evaluated to determine the sexuality of the plant and the percentage of sexual plants and apomictic plants determined (box 154).

In another optional step, if the plants are determined to be highly sexual (box 156), the steps in boxes 128 and 118-124 can be followed. Optionally these steps are performed iteratively until the desired line is produced.

If the plants are determined to be apomictic (box 160) the plants can be set up for commercial trials (box 162) and/or optional cultivar release (box 164).

Turning now to FIG. 3, in the method 200 sexual plants, optionally developed according to the method 100 of FIG. 2 are used for selection of improved traits for cultivar improvement, optional release as a cultivar, and option development of a maternal in a hybridization program. As with the steps of FIG. 2, the method of FIG. 3 comprises of a series of steps that can be performed in any order, or not at all. The various steps may occur sequentially, concurrently, iteratively or not at all. Those of skill in the art would recognize various additional and/or alternative step that may be performed.

In one optional step, one or more plants are developed as 100% sexual plants (box 210) (see for example FIG. 2 and boxes 130, 136, and 138). These 100% sexual plants may optionally be developed according to the method 100 described above in reference to FIG. 2. The various plants (box 212) may be allowed to interpollinate (box 214).

In another optional step, the seed of the various plants (box 212) may be harvested in bulk (together) (box 216) mixing the various seed together.

In a further optional step, prior to anthesis, plants may be selected, optionally those plants with superior phenotypes and/or phenotypes of interest are selected (box 218). These selected plants may then be moved into isolation (box 220).

In a further optional step, these selected plants are allowed to interpollinate in isolation (box 222). The plants may then be harvested individually (box 224), keeping the seed for different phenotypes separate.

In another optional step, a growout nursery is established for each parent (selected plant (box 218)), and progeny for each parent (box 226). In the growout nursery clones of the parent are planted alongside the progeny (box 226).

In another optional step, the parent plants are evaluated to confirm they are of obligate sexual genotypes (box 228).

In a further optional step, using the progeny, weak maternal lines are removed (box 230) and the best maternal lines or those maternal lines of interest are selected (box 232). These best or of interest maternal lines can then optionally be cloned and established together into an isolated crossing block (box 234).

The plants can then optionally be harvested by maternal parents (box 236) and seed used to established replicable, turf trials (box 236). These turf trails can then be evaluated for a period of time (box 240), optionally about 2 years.

In a further optional step, the best tillers for each maternal line are selected (box 242). Alternatively, plants with the best or other indicator of a trait of interest are selected. These plants can then be bulk harvested (box 244).

Optionally, a plant nursery can then be started with the harvested seed of the best tillers or other plants having traits of interest (box 246).

In a further optional step, the level of apomixis can be evaluated (box 248). The sexuality/level of apomixis can be evaluated at any of the above steps.

### EXAMPLES

### Example 1

In one example of breeching a highly apomixes cultivar of Kentucky bluegrass, seed from the released cultivar ‘Shamrock’ Kentucky bluegrass (PVP #9300154) was used to establish a 100-plant space plant nursery. Off-types (non-maternal) plants were selected for seed head production and commercial turfgrass characteristics. One plant selected was designated H87-337.

Seed was harvested from the plant and used to establish a space plant nursery containing 1500 plants. The population was facultative in that there were both maternal clones, apomictic, progeny and non-maternal progeny from sexual reproduction. An off-type (non-maternal clone) plant was selected out of the H87-337 plants and designated A88-690. Seed was harvested from this single plant.

Seed of A88-690 was used to establish a space plant nursery containing 1500 plants. An off-type plant was selected and designated A97-837. This plant was shown to be facultative, though highly sexual. Grow-outs showed this genotype was only about 5% sexual and 95% apomictic. So, A97-837 was maintained vegetatively in a holding nursery.

A97-837 was crossed with the released cultivar ‘Glade’, which is highly apomictic. Seed was harvested from A97-837 and used to establish a 3000 plant progeny nursery. Ninety-five percent of the plants were repeating types from the mother plant, A97-837. Five percent were determined to be hybrids. So, this genotype was 95% apomictic and 5% sexual. One of the off-type (non-maternal) plants that exhibited dark color (desirable turfgrass character) and seed yield potential (desirable seed production character) was designated AKB357.

Seed was harvested of AKB357 and used to establish a 20-plant progeny block to determine percent apomixis. The maternal line was cloned and planted bordering the 20 progeny. This ensures that the maternal clone can be identified in the progeny as well as those resulting from sexual reproduction. The plant of AKB357 was maintained in a holding nursery.

No repeating types of the maternal plant were observed in the 20-plant progeny block of AKB357. The 20 plants were completely segregated, and none looked like the maternal line, so it was determined that AKB357 was completely sexual.

The sexuality of AKB357 has been confirmed throughout the years with 1,000's of progeny screened, and no maternal clones have ever been recovered, demonstrating that this is an obligate sexual line of Kentucky bluegrass. Because of its 100% sexuality, AKB357 was put into a sexual Kentucky bluegrass breeding program and is maintained vegetatively in an isolated nursery. The plant is not allowed to flower unless it is being used for hybridizations in another area.

AKB357 was removed from the holding nursery, potted and top crossed with potted plants of the released cultivar ‘Mallard’ in a greenhouse. Seed was harvested from AKB357 and used to establish a 500-plant evaluation block. A hybrid plant from the nursery from AKB357 x ‘Mallard’ was selected and designated AKB3605. Selection was based on freedom from disease, genetic color and seed yield potential. No maternal types of the mother plant AKB357 were observed, demonstrating that it is completely sexual.

Seed of AKB3605, was used to establish a 20-plant progeny block to assess the percent apomixis. The maternal line was also cloned and planted bordering the progeny. This ensures that maternal clone can be identified in the progeny as well as those resulting from sexual reproduction. 19 of the 20 plants were harvested from the progeny block and designated breeder seed of AKB3605. The percent apomixis was calculated to be 95%.

As can be seen, with constant selection over time, completely sexual Kentucky bluegrass lines were developed and then crossed with apomict lines to get maximum segregation. Superior apomictic lines can be then be selected out of the progeny from theses crosses.

### Example 2

In this example 100% sexual genotypes to be used for the selection of improved traits for cultivar improvement and release as a cultivar and to a maternal in a hybridization program with both apomictic and sexual lines were developed.

Original parents of the following obligate sexual lines were developed as described in FIG. 2 and Example 1: AKB020—Pp H7902 x Cynthia; AKB084—Pp H7921 x Midnight; AKB276—Unique x Baron; AKB307—Unique x Baron; AKB387—Shamrock x Midnight; AKB413—Shamrock x Midnight; AKB469 AKB020 x Midnight; AKB020=Pp H7902 x Cynthia; Pp H7902=Cynthia x Donna; Pp H7921=Cynthia x Donna. For each selection cycle, apomixis/sexual level was evaluated.

Seven sexual females were planted into pots and allowed to interpollinate in a greenhouse. These sexual lines were developed as described in Example 1 and FIG. 2: AKB020, AKB084, AKB276, AKB307, AKB387, AKB413, AKB469.

Seed from the plants were harvested and across all 7 lines in bulk together and designated as AKB2568. Seed of AKB2568 was used to establish a 500-plant (space plant) evaluation block.

Twenty-five (25) plants were selected for similar morphology and seed yield potential. These were dug, potted and moved int to a greenhouse, and were allowed to interpollinate. Sixteen of the 25 plants nicked (went through anthesis at the same time). These 16 plants were allowed to set seed and were harvested individually, and the population was designated as AKB2966-1-16.

Seed from each of the 16 maternal parents was used to establish a 100-plant grow-out in the field. 100 progeny from each parent, along with clones from the maternal parent planted along both sides. Weak progeny were removed prior to flowering.

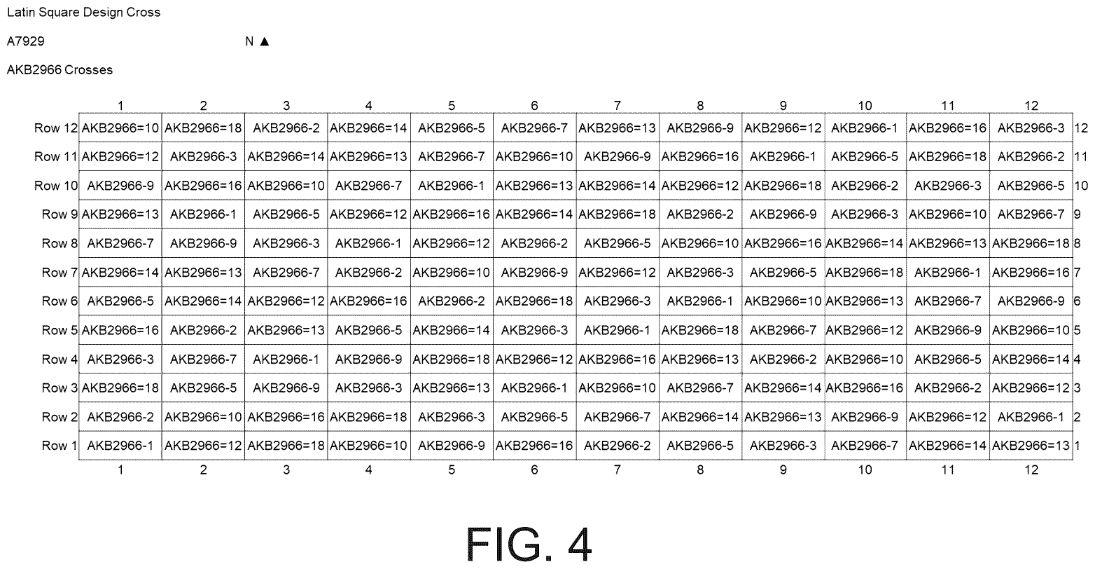

The cloned maternal lines were compared against their 100 progeny to confirm that they were obligate sexual lines and to determine the fitness of each maternal line by studying the progeny produced. Maternal lines producing inferior progeny were dropped. Progeny from the 12 best maternal lines were harvested. Each line was kept separate. Clones from the 12 best performing maternal lines used to establish a Diallel cross, as shown in FIG. 4.

Crossing block of 12 best plants performing plants established (7 plants each line=84 clones) and designated AKB3284. The crossing block was then harvested by lines.

Next, the AKB3284-1-12 progenies were established in a turf trial in Salem, NJ. The trial allowed to be subjected to disease and summer stresses. Fifty (50) tillers surviving from each progeny line were selected and designated as AKB419. AKB419 was harvested by bulk. Then a 3,000 space-plant nursery was established to determine whether apomixis was completely absent. The plants were returned to or established in an isolated crossing block. The population was designated AKB4191. The apomixis level was evaluated and it was confirmed that the population was completely sexual.

### Example 3

Various characteristics of plants can be measured/observed data for the various cultivars is listed in Tables 1-5. Table 1 shows data for development of various cultivars according to the methods described herein, 16PVPPPA; Table 2 shows data for AKB413 and Mallard cross; Table 3 shows data for AKB and Mallard crosses; Table 4 shows data for AKB and Diva crosses; and Table 5 shows data for AKB and Bonaire crosses. Table 1 shows data for apomictic plant developed from the hybridizations of sexual Kentucky Bluegrass with apomictic Kentucky Bluegrass plants. Tables 2-5 show subsets of data from Table 1, describing the morphological characteristics of specific hybridizations. In Table 2 the bottom row represents cultivar AKB413 is the sexual mother line; while the top rows are apomictic populations from crossing a sexual plant (AKB413) with an apomictic male plant (as described in FIG. 3) to develop a highly apomictic cultivar from the sexual plant. In Table 3 the two apomictic cultivars (AKB1658 and AKB3605) were developed from the crossing of the sexual plant (AKB357) with an apomictic male parent (Mallard).

Additionally, the plants labeled as sexual in Tables 1-5, are plants developed as described in FIG. 2; where a sexual plant was hybridized with an apomictic male parent (an apomictic cultivar). It is from these hybridizations that new apomictic plants were developed.

As shown in the various tables, the disclosed methods allow for crossing apomictic parents and developing completely sexual plants. Further, crossing the completely sexual plants back with an apomictic parent can allow for developing new cultivars.

The measured characteristics may include:

Genetic Color—the measure of the amount of lightness or darkness of green color. Recorded as a 1-9 subjective rating where 9=dark.

Growth Habit—the degree of erectness of a single plant. 1=prostrate (flat), 2=semi-prostrate, 3=horizontal, 7=semi-erect, 9=erect.

Inflorescence—the flowering portion of a grass plant (in ryegrass the inflorescence is a spike).

Spike Length—measured from the upper most node to the apex of the inflorescence.

Node—the joint on a grass culm; a swollen region on the stem.

Leaf Blade—the flattened portion of a grass leaf located above the leaf sheath.

Leaf Blade Length—the length of the leaf blade; measured on the first leaf subtending the flag leaf in cm.

Leaf Blade Width—measure of the width of the first blade subtending the flag leaf in mm taken 1 cm from the collar.

Leaf Blade Height—the height of the leaf blade from the ground to the collar in cm.

Leaf Sheath Length—the length of the leaf sheath; measured on the first leaf subtending the flag leaf in cm.

Flag Leaf—the first leaf blade subtending the inflorescence.

Flag Leaf Length—the total length of a flag leaf which includes the sheath and blade. Measured from the uppermost node to the end of the upper most blade in cm.

Flag Leaf Width—the measure of the width of the flag leaf blade taken 1 cm from the collar of the flag leaf in mm.

Flag Leaf Height—the height of the flag leaf, measured from the ground to the collar of the flag leaf in cm.

Flag Leaf Sheath Length—the sheath length of the flag leaf, measured from the node to the collar in cm.

Mature Plant Height—the height in cm of a mature plant from the ground to the apex of a mature inflorescence.

Glume—the first pair of bracts at the base of a spikelet.

Spikelet—the basic unit of a grass inflorescence, includes glumes, lemmas, paleas and reproductive organs.

Floret—the portion of the spikelet that may include lemma, palea and reproductive organs.

Lemma—an odd veined bract above the glumes.

Palea—the two veined bract above the glumes and lemma, subtending the reproductive organs.

Seed Size—the relative size of seeds usually measured by determining the number of seeds per pound.

1000 seed weight—the weight of 1,000 whole seeds.

Turfgrass Density—the number of tillers per unit area of a turfgrass sward.

Turf Color—a visual and digital analysis score of the turfgrass community, when visual the color is measured on a scale of 1-9 with 9 being dark.

Turf Quality—the degree to which a turf conforms to a standard of uniformity, density, texture, growth habit, color and is generally taken as subjective data on a 1-9 scale with 9 being the best quality.

Although the disclosure has been described with references to various embodiments, persons skilled in the art will recognize that changes may be made in form and detail without departing from the spirit and scope of this disclosure.

What is claimed is:

1. A method for producing hybrid seed comprising:
crossing a maternal completely sexual plant with a paternal facultative or apomictic cultivar to create a first hybrid generation;
growing the first hybrid generation;
selecting phenotypes of interest from plant of the first hybrid generation;
harvest selected phenotype plants individually; and
grow individually the selected phenotype plants along side a clone of a maternal parent of the select phenotype plant to create a second hybrid generation.

2. The method of claim 1, further comprising determining the percent sexuality of the second hybrid generation.

3. The method of claim 2, further comprising releasing a cultivar for the second hybrid generation when the second hybrid generation is apomictic.

4. The method of claim 1, wherein the maternal completely sexual plant is an obligate sexual Kentucky bluegrass.

5. The method of claim 4, wherein the paternal facultative cultivar is a plant having desired characteristics.

6. The method of claim 1, wherein the apomictic mechanism from the paternal facultative or apomictic cultivar is transferred to the first and second hybrid generations.

7. The method of claim 1, wherein the paternal facultative or apomictic cultivar is an apomictic cultivar.

8. A seed of a plant produced via the method of claim 1.

9. A grass plant produced by growing the seed of claim 8.

10. A grass plant having all the physiological and morphological characteristics of the grass plant of claim 9.
